(19) United States
(12) Patent Application Publication
(10) Pub. No.: US 2010/0030030 A1
(43) Pub. Date: Feb. 4, 2010

(54) ENDOSCOPE CHANNEL TUBE AND METHOD OF PRODUCING THE SAME

(76) Inventors: Noriyuki ONOBORI, Kanagawa (JP); Ichiro Amimori, Kanagawa (JP); Takayuki Nakamura, Kanagawa (JP)

Correspondence Address: BIRCH STEWART KOLASCH & BIRCH, PO BOX 747, FALLS CHURCH, VA, 22040-0747, US

(21) Appl. No.: 12/510,737

(22) Filed: Jul. 28, 2009

(30) Foreign Application Priority Data
Jul. 29, 2008 (JP) 2008-194752
Jul. 29, 2008 (JP) 2008-194753

(51) Int. Cl. A61B 1/018; B29C 69/00

(52) U.S. Cl. 600/153; 29/527.1

(57) ABSTRACT

A forceps channel tube is composed of a tube body, a reinforcing tape and a protective layer. The reinforcing tape includes a reinforcement layer and an adhesive layer. The reinforcement layer has a plurality of strip-shaped high and low rigidity portions arranged alternately. The reinforcing tape is wrapped around an outer surface of the tube body in a manner that each of the high and low rigidity portions encircles the tube body along the circumferential direction, and fixed thereto by the adhesive layer. The reinforcing tape provides the tube body with rigidity anisotropy that increases a first rigidity against force in a radial direction of the tube body to exceed a second rigidity against bending force.

### FIELD OF THE INVENTION

The present invention relates to an endoscope channel tube and a method of producing the same.

### BACKGROUND OF THE INVENTION

A standard endoscope generally has an insertion section to be inserted into a patient's body cavity and a handling section for operating the insertion section. The insertion section is composed of a flexible portion, a bending portion and a distal portion. In diagnostic examination and surgical operation using the endoscope, various medical treatments involving extraction and resection of body tissue are performed with a snare loop or such a medical instrument inserted through a forceps channel tube. The forceps channel tube runs entirely through the insertion section.

The forceps channel tube is generally made of fluorine resin to improve the smoothness of a tube interior surface that facilitates the insertion of the medical instrument, and improve the contamination and chemical resistance of the tube. During diagnostic examination, the forceps channel tube elastically bends in accordance with a bending action of the bending portion. At an acute bending angle, however, the forceps channel tube may have a tube kink (or buckling) in which the forceps channel tube collapses radially inwardly. The tube kink makes it difficult to advance the medical instrument in the forceps channel tube, and at worst, the forceps channel tube may break during the passage of the medical instrument. In view of this, most forceps channel tubes are reinforced to prevent the tube kink during the bending.

Japanese Patent Laid-open Publication No. 04-341836 discloses a flexible tube to be used as the forceps channel tube. This flexible tube has a fluororesin tube reinforced by an outer layer having an embedded metal mesh. Japanese Patent Laid-open Publication No. 05-228106 discloses an endoscope channel tube composed of a fluororesin tube with a spiral groove on its outer surface and a metal spring fitted in the spiral groove to reinforce the fluororesin tube.

U.S. Pat. No. 5,529,820 (corresponding to Japanese Patent Laid-open Publication No. 06-270301) discloses a flexible tube composed of a porous PTFE (polytetrafluoroethylene) substrate tube filled with silicone rubber, an airtight second layer on the substrate tube, and a reinforcement layer of porous PTFE film on the second layer. This reinforcement layer is wrapped around the second layer, and fixed thereon with adhesive.

While the outer layer and the reinforcement layer disclosed in the Publication No. 04-341836 and U.S. Pat. No. 5,529,820 can enhance tubes' rigidity against force in the radial direction and prevent the collapse of the tubes, they also enhance tubes' rigidity against bending force. Such enhanced rigidity against the bending force impedes the tubes from bending, and reduces the insertability and the operability of the endoscope. As for the tube of the Publication No. 05-228106, on the other hand, the metal spring may possibly come off from the groove in a repetitive bending action of the tube.

### SUMMARY OF THE INVENTION

In view of the foregoing, it is an object of the present invention to provide a durable endoscope channel tube which is easily bendable but hard to collapse, and a method of producing this endoscope tube.

In order to achieve the above and other objects, the endoscope tube according to the present invention includes a flexible tube body, a reinforcing tape wound around and fixed on an outer surface of the tube body, and a protective layer for covering the reinforcing tape. The reinforcing tape has an adhesive layer sticking to the outer surface of the tube body and a reinforcement layer formed on the adhesive layer. This reinforcing tape provides the tube body with rigidity anisotropy that enhances a first rigidity against force in a radial direction of the tube body to exceed a second rigidity against bending force.

The tube body preferably has a smoothed surface. The reinforcement layer preferably has a low rigidity portion and a plurality of strip-shaped high rigidity portions encircling the tube body along a circumferential direction thereof.

In a preferred embodiment of the present invention, the high rigidity portions are first fibers extending lengthwise of the reinforcing tape, and the low rigidity portion is a resin layer that holds the first fibers. The reinforcement layer further includes second fibers extending crosswise of the reinforcing tape. These second fibers are worked with the first fibers to form a net, which is at least partially embedded in the resin layer.

It is preferred to make the first and second fibers from the same material. A preferred material is polyester.

To enhance the first rigidity, the first fibers may be made of a material having higher rigidity than the second fibers. To further enhance the first rigidity, the first fibers may be made thicker than the second fibers, or greater in number than the second fibers. The tube body is a forceps channel tube.

In another preferred embodiment, the reinforcement layer is composed of strip-shaped high rigidity portions made of hard resin and strip-shaped low rigidity portions made of soft resin with lower rigidity than the hard resin. These high and low rigidity portions are arranged alternately along a crosswise direction of the reinforcing tape, and extend in a lengthwise direction of the reinforcing tape.

The hard resin and soft resin are made of the same main material. Additionally, the high rigidity portions are wider in an axial direction of the tube body than the low rigidity portions.

A method of producing an endoscope channel tube according to the present invention includes the steps of fabricating a reinforcement layer, making a reinforcing sheet, slitting the reinforcing sheet in a first direction to make a reinforcing tape with a constant width, winding the reinforcing tape, and forming a protective layer for covering the reinforcing tape. In the reinforcing sheet making step, an adhesive layer is formed on an undersurface of the reinforcement layer. In the reinforcing tape winding step, the reinforcing tape is wound around the tube body with the adhesive layer facing inward. The reinforcing tape provides a tube body with rigidity anisotropy that enhances a first rigidity against force in a radial direction of the tube body to exceed a second rigidity against bending force.

It is preferred to insert a core into the tube body prior to the reinforcing tape winding step, and remove this core from the tube body subsequent to the protective layer forming step. It is also preferred to conduct surface treatment, prior to the reinforcing tape winding step, for smoothing an outer surface of the tube body. The surface treatment is one of chemical etching, plasma etching and mechanical polishing. It is also preferred to heat the reinforcement layer in the reinforcing tape winding step.

In a preferred embodiment, the reinforcement layer fabricating step includes the steps of attaching first fibers extending in the first direction to a layer of resin monomer at predetermined pitches, and curing the resin monomer to fix the first fibers.

Preferably, the reinforcement layer fabricating step includes the step of forming a net, attaching a net and fixing the net. In the net forming step, the first fibers extending in the first direction are worked with second fibers extending in a second direction crosswise to the first direction to form the net. In the net attaching step, the net is attached to a layer of resin monomer. In the net fixing step, the resin monomer is cured to fix the first fibers.

The first fibers are thicker than the second fibers. The reinforcement layer fabricating step preferably includes the step of heat compression to flatten the net, prior to the net attaching step.

In another preferred embodiment, the reinforcement layer has a plurality of strip-shaped high and low rigidity portions which are arranged alternately in a second direction crosswise to the first direction and extend in the first direction. The high rigidity portions are made of hard resin and the low rigidity portions are made of soft resin having lower rigidity than the hard resin.

The reinforcement layer fabricating step includes the steps of molding the hard resin, curing the soft resin, and removing a part of a hard resin molding. In the hard resin molding step, the hard resin is molded using a casting mold with a cavity. In the soft resin molding step, molten soft resin is poured into a cavity of the hard resin molding, and the soft resin is cured therein. In the removing step, one surface of the hard resin molding is removed until the interface of the hard and soft resin is revealed.

According to the present invention, the reinforcing tape wrapped spirally around the outer surface of the tube body increases a first rigidity against force in a radial direction of the tube body to exceed a second rigidity against bending force. The endoscope channel tube is thus easily bendable, but hard to collapse. It is therefore possible to prevent the collapse of the endoscope channel tube upon bending without spoiling the good operability of the endoscope.

Fixed to the outer surface of the tube body by the adhesive layer, the reinforcing tape does not come off from the tube body even in a repetitive bending action of the endoscope channel tube. Accordingly, the endoscope channel tube can be ever more durable.

### BRIEF DESCRIPTION OF THE DRAWINGS

The above objects and advantages of the present invention will become more apparent from the following detailed description when read in connection with the accompanying drawings, in which:

FIG. 1 is a schematic view of an endoscope;

FIG. 2 is a perspective view of a forceps channel tube according to the present invention;

FIG. 3 is a flow chart for the production of the forceps channel tube;

FIG. 4A to FIG. 4F are cross sectional views illustrating a process of producing a reinforcing tape;

FIG. 5 is a perspective view of a forceps channel tube according to the second embodiment; and

FIG. 6A to FIG. 6D are cross sectional views illustrating a process of producing the reinforcing tape shown in FIG. 5.

### DETAILED DESCRIPTION OF THE PREFERRED EMBODIMENTS

### First Embodiment

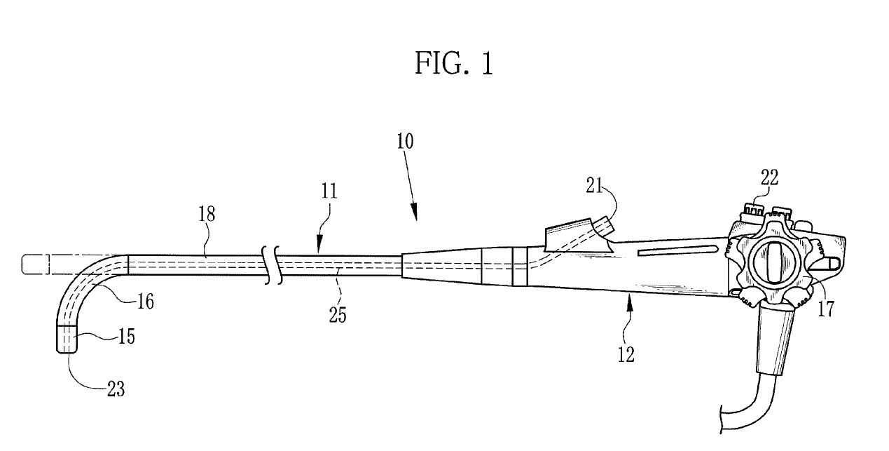

Referring to FIG. 1, an endoscope 10 includes a flexible insertion section 11 to be inserted into a body cavity, and a handling section 12 connected to a proximal end of the insertion section 11.

At a distal tip of the insertion section 11, there is provided a distal portion 15 containing a CCD (not shown) for capturing an image in the body cavity. The distal portion 15 is connected to the insertion section 11 via a bending portion 16 composed of a plurality of annular joint pieces which are pivotally linked adjacent to one another. The bending portion 16 bends up, down, right and left in accordance with the operation of an angle knob 17 provided on the handling section 12, and orients the distal portion 15 in a desired direction.

The handling section 12 is provided with a forceps insertion port 21 to insert a snare loop or such a medical instrument, and an air/water feed button 22 to be pressed for insufflation and irrigation of air and rinse water from an air/water feeding device (not shown). On a front end surface of the distal portion 15, a forceps outlet port 23 opens. The forceps outlet port 23 is coupled to the forceps insertion port 21 via a forceps channel tube 25. The forceps channel tube 25 runs through the handling section 12, the insertion section 11, the bending portion 16 and the distal portion 15. The medical instrument being inserted through the forceps insertion port 21 passes entirely through the forceps channel tube 25, and projects into the body cavity from the forceps outlet port 23.

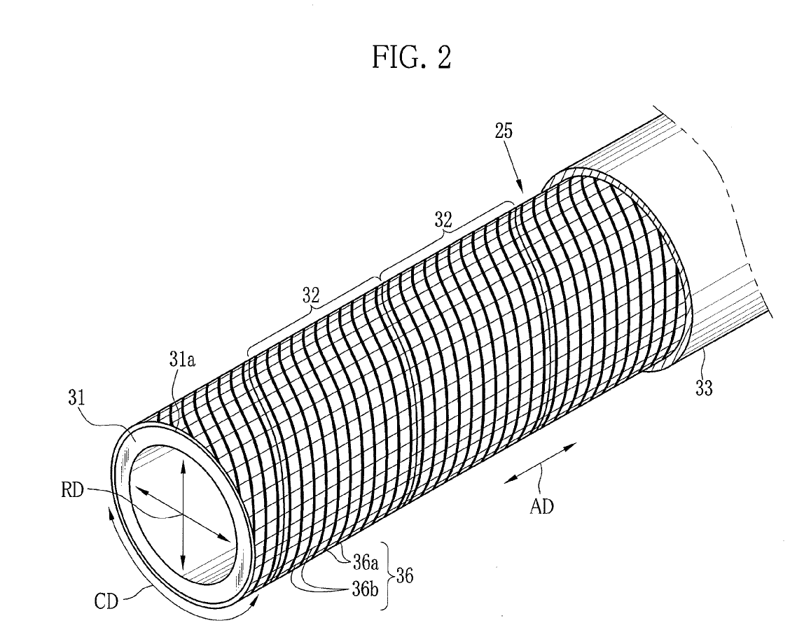

As shown in FIG. 2, the forceps channel tube 25 includes a tube body 31, a reinforcing tape 32 and a protective layer 33. The tube body 31 is made of a flexible material, such as fluorine resin (fluororesin). The protective layer 33 is made of, for example, polyurethane, and covers the entire length of the reinforcing tape 32.

The reinforcing tape 32 is constant in width (for example, 10 mm), and wrapped spirally around an outer surface 31a of the tube body 31 and fixed thereto. The reinforcing tape 32 provides the tube body 31 with rigidity anisotropy that increases the rigidity against force in a radial direction RD (hereinafter, radial rigidity) to exceed the rigidity against bending force (hereinafter, bending rigidity). With the reinforcing tape 32, the radial rigidity of the tube body 31 is preferably enhanced to 1.1 to 1000 times, and more preferably, 2 to 1000 times as much as the bending rigidity.

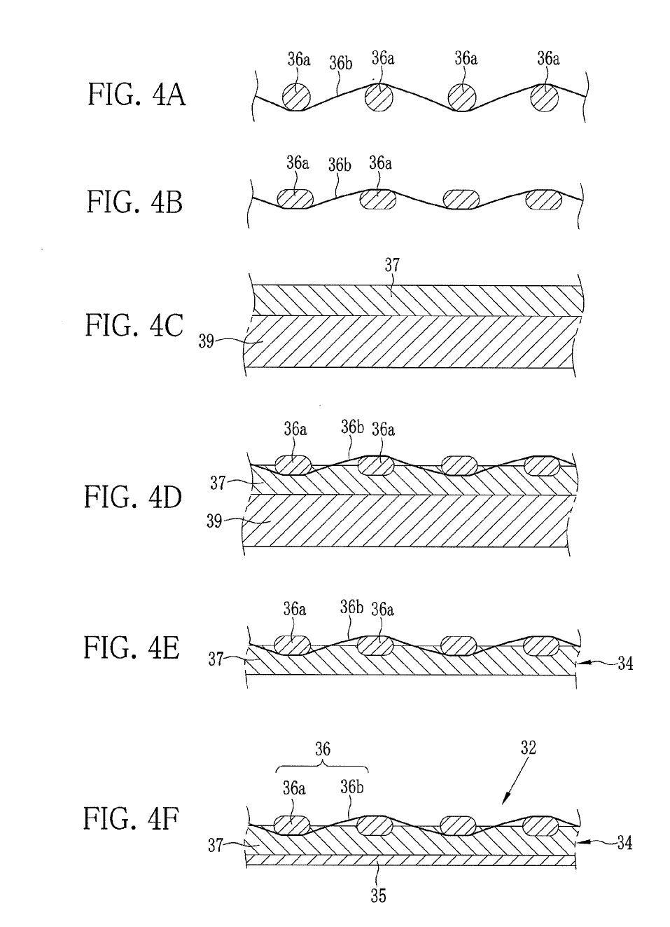

As shown in FIG. 4F, the reinforcing tape 32 is composed of a reinforcement layer 34 and an adhesive layer 35. The reinforcement layer 34 includes a reinforcing net 36 and a resin layer 37. The reinforcing net 36 is made by working or knitting circumferential reinforcing fibers 36a and axial reinforcing fibers 36b.

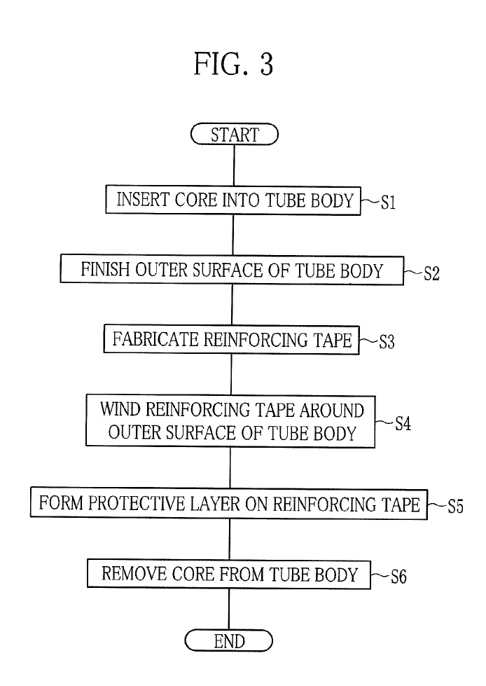

As shown in a flow chart of FIG. 3, in producing the forceps channel tube 25, a metal core (not shown) is inserted into the tube body 31 (S1). This metal core prevents the deformation of the tube body 31 as the reinforcing tape 32 is wrapped around the tube body 31. In the case of forming the tube body 31 together with the metal core by an extrusion molding method, however, the step S1 may be omitted.

The outer surface 31a of the tube body 31 is then finished by chemical etching using a sodium metal solution or the like (S2). This surface treatment smoothes the outer surface 31a, and improves adhesion to the reinforcing tape 32. The outer surface 31a may also be finished by plasma etching or mechanical polishing. In terms of physical and chemical stability of the outer surface 31a, however, the chemical etching or the plasma etching is advantageous. This surface treatment can be performed immediately before the reinforcing tape 32 is wrapped and fixed to the outer surface 31a.

The next step is the fabrication of the reinforcing tape 32 composed of the reinforcement layer 34 and the adhesive layer 35 (S3). The reinforcing tape 32 is then wrapped spirally around the outer surface 31a of the tube body 31, and fixed thereto by the adhesive layer 35 (S4). This step 4 is preferably carried out with using an automatic tape winding machine, and the reinforcement layer 34 may be heated during this step. In doing so, the deformation of the reinforcement layer 34 can be avoided, and a stable wrapping pattern of the reinforcing tape 32 can be created. It is also preferred to firstly wrap the reinforcing tape 32 to extend a short distance past the both ends of the tube body 31, and then trim these extended portions of the reinforcing tape 32. This process allows for wrapping the reinforcing tape 32 throughout the length of the tube body 31. The automatic tape winding machine may be, for example, the coil winder shown in FIG. 7 of the aforesaid Publication No. 05-228106, or such a machine to rotate the forceps channel tube 25 around the axis and wrap the reinforcing tape 32 spirally around the outer surface 31a. The reinforcing tape 32 is wrapped into a single layer, so that the adjacent turns of the reinforcing tape 32 touch each other on the side edges. Alternatively, the side edge may overlap the side edge of the previous turn, or spacing may be left between the side edges. To enhance the strength, it is possible to wrap the reinforcing tape 32 twice or more times into a multi layer.

After the wrapping of the reinforcing tape 32 around the tube body 31, a coating layer of polyurethane or the like is applied over the reinforcing tape 32 to form the protective layer 33 (S5). The protective layer 33 smoothes the surface of the reinforcing tape 32, and improves the slideability of the forceps channel tube 25. In the end, the metal core is removed or pulled out from the tube body 31 (S6). Alternatively, in place of the polyurethane coating layer, a heat-shrinkable tube may be used which is placed over the reinforcing tape 32 and heat-shrunk to coat the reinforcing tape 32. Although the reinforcing tape 32 is fabricated (S3) between the surface treatment (S2) and the wrapping process (S4) for convenience sake, it is better to prepare the reinforcing tape 32 in advance before the production of the forceps channel tube 25.

Hereafter, with reference to FIG. 4A to FIG. 4F, the fabrication step (S3) of the reinforcing tape 32 is explained. Firstly, the circumferential reinforcing fibers 36a are knitted with the axial reinforcing fibers 36b in regular pitches to produce the lengthy (for example, 10 m long) reinforcing net 36 shown in FIG. 4A. Each circumferential reinforcing fiber 36a intersects with the axial reinforcing fibers 36b at an angle of, for example, 70-90°. These circumferential and axial reinforcing fibers 36a, 36b are made of the same material (polyester, for example). To prevent the collapse of the tube body 31, the circumferential reinforcing fibers 36a are thick in diameter. Optionally, carbon powder or the like may be added to provide extra rigidity of the circumferential reinforcing fibers 36a. The axial reinforcing fibers 36b, by contrast, are thin in diameter to facilitate the bending action. A resin layer 37 is made of UV curing resin having lower rigidity than the circumferential reinforcing fibers 36a (e.g., polyester).

The reinforcing net 36 is then pressed thermally (heat compression) to flatten the upper and under sides of the circumferential reinforcing fibers 36a.

Then, as shown in FIG. 4C, resin monomer is applied on a base material 39 of PET to form a resin layer 37, and as shown in FIG. 4D, the reinforcing net 36 is placed on a top surface of the resin layer. As the resin monomer fills the gaps between the circumferential and axial reinforcing fibers 36a, 36b, the resin layer 37 is hardened by UV-curing so as to fix the reinforcing net 36. Then, as shown in FIG. 4E, the base material 39 is removed. Alternatively, the reinforcement layer 34 may be formed by a dip molding process in which the reinforcing net 36 is firstly dipped in resin monomer solution, and then the resin monomer on the reinforcing net 36 is hardened by UV-curing.

After the removal of the base material 39, the adhesive layer 35 is formed on an undersurface of the resin layer 37 to make a lengthy reinforcing sheet shown in FIG. 4F. In the end, the reinforcing sheet is slit along, for example, the circumferential reinforcing fibers 36a to obtain the reinforcing tape 32 with 10 mm width. Accordingly, on the reinforcing tape 32, the circumferential reinforcing fibers 36a extend in the lengthwise direction, and the axial reinforcing fibers 36b extend in the crosswise direction. Nonetheless, the axial reinforcing fibers 36b can be somewhat inclined to the crosswise direction. Moreover, the circumferential and axial reinforcing fibers 36a, 36b may be both inclined to the crosswise direction.

Extending in the lengthwise direction of the reinforcing tape 32, each of the circumferential reinforcing fibers 36a forms a spiral along a circumferential direction CD of the tube body 31 as the reinforcing tape 32 is wrapped. On the other hand, the axial reinforcing fibers 36b of less rigidity extend discontinuously along an axial direction AD of the tube body 31. This configuration provides the rigidity anisotropy, which makes the forceps channel tube 25 free from collapsing upon bending, without spoiling the good operability. The resin layer 37 cannot be a serious load to the bending action because it does not have much rigidity. Additionally, since the adhesive layer 35 sticks to the tube body 31, the reinforcing tape 32 does not come off during the bending action.

To farther improve the radial rigidity, the circumferential reinforcing fibers 36a can be made of a more rigid material than the axial reinforcing fibers 36b. Instead of or in addition to this, the circumferential reinforcing fibers 36a may be arranged at smaller pitches than the axial reinforcing fibers 36b, so as to increase the number of circumferential reinforcing fibers 36a per unit area.

One thing to note is that the axial reinforcing fibers 36b only function as a support for the circumferential reinforcing fibers 36a before the reinforcing net 36 is fixed to the resin layer 37, and do not contribute to prevent the collapse of the forceps channel tube 25. Rather, the axial reinforcing fibers 36b slightly enhance the bending rigidity of the tube body 31, and makes the forceps channel tube 25 somewhat difficult to bend. It is therefore preferred to minimize the number of the axial reinforcing fibers 36b or to reduce the diameter of each axial reinforcing fiber 36b. Where possible, it may be better not to use the axial reinforcing fibers 36b.

### Second Embodiment

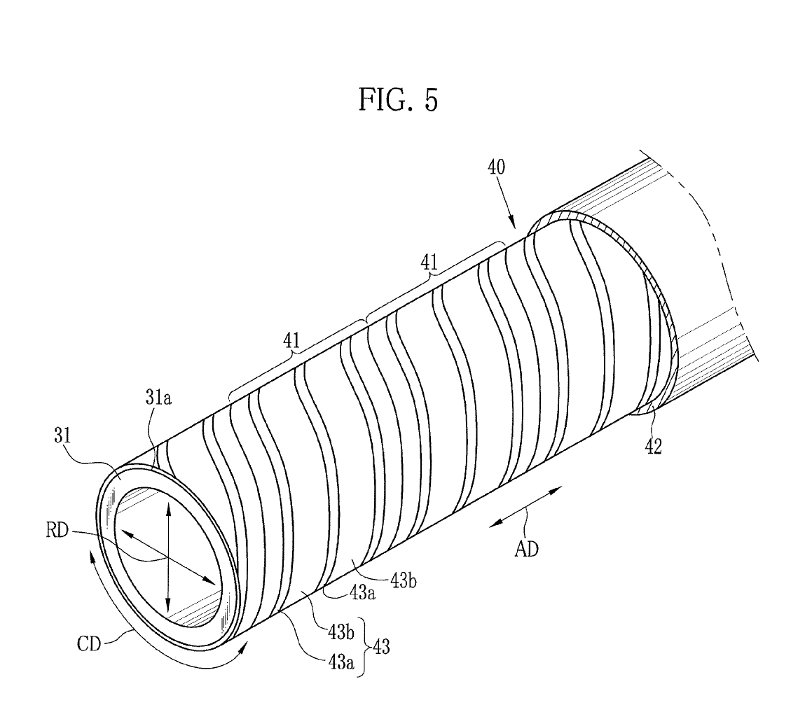

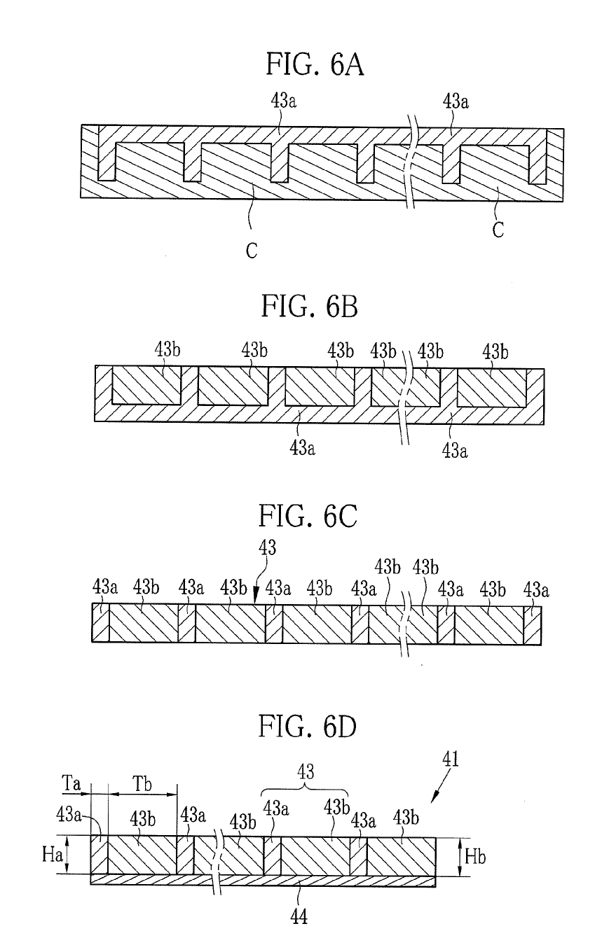

Next, with reference to FIG. 5 and FIG. 6A to FIG. 6D, a second embodiment of the present invention is described. Hereafter, elements similar to those in the first embodiment are designated by the same reference numerals, and detailed explanation thereof is omitted. As shown in FIG. 5, a forceps channel tube 40 includes the tube body 31, a reinforcing tape 41 and a protective layer 42. The reinforcing tape 41 is attached, in a spiral configuration, to the outer surface 31a of the tube body 31.

As shown in FIG. 6D, the reinforcing tape 41 is composed of a reinforcement layer 43 and an adhesive layer 44. The reinforcement layer 43 includes a plurality of strip-shaped hard and soft resin portions 43a, 43b. The hard resin portions 43a are more rigid than the soft resin portions 43b. The adhesive layer 44 is formed on an undersurface of the reinforcement layer 43. The soft resin portions 43b have a width Tb that is wider than a width Ta of the hard resin portions 43a. This configuration provides the rigidity anisotropy to the reinforcing tape 41. With the reinforcing tape 41, the radial rigidity of the tube body 31 is preferably enhanced to 1.1 to 1000 times, and more preferably, 2 to 1000 times as much as the bending rigidity.

In fabricating the reinforcing tape 41, as shown in FIG. 6A, molten hard resin is poured into cavities of a lengthy casting mold C (10 m long, for example), and cured therein to mold the hard resin portions 43a. As shown in FIG. 6B, this hard resin molding is removed from the casting mold C and turned over, and then molten soft resin (having a lower softening temperature than the hard resin portions 43a) is poured into cavities of the hard resin molding, and cured therein to mold the soft resin portions 43b. As shown in FIG. 6C, a bottom surface of the hard resin molding is removed by polishing or the like until the interfaces between the hard and soft resin portions 43a, 43b are revealed. The reinforcement layer 43 is thereby obtained. Then, the adhesive layer 44 is attached on the undersurface of the reinforcement layer 43 to make a lengthy reinforcing sheet shown in FIG. 6D. In the end, the reinforcing sheet is slit along the hard and soft resin portions 43a, 43b to obtain the reinforcing tape 41 having a width of about 10 mm.

The hard and soft resin portions 43a, 43b extend lengthwise of the reinforcing tape 41. As shown in FIG. 5, this reinforcing tape 41 is wrapped spirally around the outer surface 31a of the tube body 31, and fixed thereto by the adhesive layer 44.

Extending in the lengthwise direction of the reinforcing tape 41, each of the hard resin portions 43a forms a spiral along the length of the tube body 31 as the reinforcing tape 41 is wrapped. The hard resin portions 43a prevent the collapse of the tube body 31. On the other hand, the relatively-wide soft resin portions 43b are arranged alternately with the relatively-narrow hard resin portions 43a in the axial direction AD of the tube body 31. Upon bending of the forceps channel tube 25, the soft resin portions 43b deform (shrink or stretch). At the same instance, the hard resin portions 43a barely deform due to the narrow width, and cause little resistance in the bending action. Therefore, the forceps channel tube 25 can resist the collapse during bending, but still ensures good operability.

Modulus of section of an object increases as the object grows taller to increase its radial rigidity. Therefore, the hard resin portions 43a may have a height Ha that is higher than a height Hb of the soft resin portions 43b. The height ratio may be determined by experiment.

The hard and soft resin portions 43a, 43b can be made of the same material. In this case, an additive is added to one of the material to change the rigidity. By way of example, polyester may be the material for the hard and soft resin, and carbon powder may be added to the hard resin to enhance the rigidity.

Although the reinforcing tapes 32, 41 are wrapped throughout the length in the axial direction AD of the tube body 31, they may be wrapped around tube body 31 only within the bending portion 16.

While the above embodiments use the tube body 31 made of fluororesin, the tube body 31 may be made of any conventional flexible material other than the fluororesin.

The rigidity anisotropy of the endoscope is a product of high and low rigidity portions that are arranged alternately along the axial direction AD while encircling the tube body in the circumferential direction CD. While the first and second embodiments use the circumferential reinforcing fibers 36a and the hard resin portions 43a as the high rigidity portions, and the resin layer 37 and the soft resin portions 43b as the soft rigidity portions, the high and low rigidity portions are not limited thereto.

While the above embodiments are directed to the forceps channel tube, the present invention is also applicable to the air/water channel tube and other endoscope tubes.

Although the present invention has been fully described by the way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. An endoscope channel tube comprising:
a flexible tube body;
a reinforcing tape wound around and fixed on an outer surface of said tube body, said reinforcing tape having an adhesive layer sticking to said outer surface of said tube body and a reinforcement layer formed on said adhesive layer, said reinforcing tape providing said tube body with rigidity anisotropy that increases a first rigidity against force in a radial direction of said tube body to exceed a second rigidity against bending force; and
a protective layer for covering said reinforcing tape.

2. The endoscope channel tube of claim 1, wherein said tube body has a smoothed surface.

3. The endoscope channel tube of claim 1, wherein said reinforcement layer has a low rigidity portion and a plurality of strip-shaped high rigidity portions encircling said tube body along a circumferential direction thereof.

4. The endoscope channel tube of claim 3, wherein said high rigidity portions are first fibers extending lengthwise of said reinforcing tape, and said low rigidity portion is a resin layer holding said first fibers.

5. The endoscope channel tube of claim 4, wherein said reinforcement layer further includes second fibers extending crosswise of said reinforcing tape, said second fibers are worked with said first fibers to form a net, and said net is at least partially embedded in said resin layer.

6. The endoscope channel tube of claim 5, wherein said first and second fibers are made of the same material.

7. The endoscope channel tube of claim 6, wherein said material is polyester.

8. The endoscope channel tube of claim 5, wherein said first fibers are made of a material having higher rigidity than said second fibers.

9. The endoscope channel tube of claim 5, wherein said first fibers are thicker than said second fibers, or greater in number than said second fibers.

10. The endoscope channel tube of claim 1, wherein said tube body is a forceps channel tube.

11. The endoscope channel tube of claim 1, wherein said reinforcement layer has strip-shaped high rigidity portions made of hard resin and strip-shaped low rigidity portions made of soft resin with lower rigidity than said hard resin, said high and low rigidity portions being arranged alternately along a crosswise direction of said reinforcing tape and extending in a lengthwise direction of said reinforcing tape.

12. The endoscope channel tube of claim 11, wherein said hard resin and soft resin are made of the same main material.

13. The endoscope channel tube of claim 11, wherein said high rigidity portions are wider in an axial direction of said tube body than said low rigidity portions.

14. A method of producing an endoscope channel tube having a flexible tube body comprising the steps of:
fabricating a reinforcement layer;
forming an adhesive layer on an undersurface of said reinforcement layer to make a reinforcing sheet;
slitting said reinforcing sheet in a first direction to make a reinforcing tape with a constant width;
winding said reinforcing tape around said tube body with said adhesive layer facing inward, said reinforcing tape providing said tube body with rigidity anisotropy that increases a first rigidity against force in a radial direction of said tube body to exceed a second rigidity against bending force; and
forming a protective layer for covering said reinforcing tape.

15. The method of producing the endoscope channel tube of claim 14, further comprising the steps of:
inserting a core into said tube body, prior to said reinforcing tape winding step; and
removing said core from said tube body, subsequent to said protective layer forming step.

16. The method of producing the endoscope channel tube of claim 14, further comprising the step of surface treatment for smoothing an outer surface of said tube body, prior to said reinforcing tape winding step.

17. The method of producing the endoscope channel tube of claim 16, wherein said surface treatment is one of chemical etching, plasma etching and mechanical polishing.

18. The method of producing the endoscope channel tube of claim 14, wherein said reinforcement layer is heated while said reinforcing tape is wound in said reinforcing tape winding step.

19. The method of producing the endoscope channel tube of claim 14, wherein said reinforcement layer fabricating step comprises the steps of:
attaching first fibers extending in said first direction to a layer of resin monomer at predetermined pitches, and
curing said resin monomer to fix said first fibers.

20. The method of producing the endoscope channel tube of claim 14, wherein said reinforcement layer fabricating step comprises the steps of:
working said first fibers extending in said first direction with second fibers extending in a second direction crosswise to said first direction to form a net;
attaching said net to a layer of resin monomer; and
curing said resin monomer to fix said first fibers.

21. The method of producing the endoscope channel tube of claim 20, wherein said first fibers are thicker than said second fibers.

22. The method of producing the endoscope channel tube of claim 21, wherein said reinforcement layer fabricating step further comprises the step of heat compression to flatten said net, prior to said net attaching step.

23. The method of producing the endoscope channel tube of claim 14, wherein said reinforcement layer has a plurality of strip-shaped high and low rigidity portions being arranged alternately in a second direction crosswise to said first direction and extending in said first direction, said high rigidity portions are made of hard resin and said low rigidity portions are made of soft resin having lower rigidity than said hard resin.

24. The method of producing the endoscope channel tube of claim 23, wherein said reinforcement layer fabricating step comprises the steps of:
molding said hard resin using a casting mold with a cavity;
pouring molten soft resin into a cavity of said hard resin molding, and curing said soft resin therein; and
removing a surface of said hard resin molding until the interface of said hard and soft resin is revealed.
